SEQUENCE LISTING (1 sequence)

SEQ ID NO: 1 - protein, 8 aa, Artificial Sequence
  Synthetic peptide
QWAVGHLM

(19) United States
(12) Patent Application Publication
(10) Pub. No.: US 2017/0121368 A1
(43) Pub. Date: May 4, 2017

(54) NOVEL COMPOUNDS COMPRISING A BOMBESIN DERIVATIVE, A PROCESS FOR THE PREPARATION THEREOF AND A NUCLEAR MOLECULAR IMAGING AGENT COMPRISING THE SAME

(71) Applicant: Korea Institute of Radiological & Medical Sciences, Seoul (KR)

(72) Inventors: Yong Jin Lee, Seoul (KR); Joo Hyun Kang, Seoul (KR); Kwang Il Kim, Seoul (KR); Min Hwan Kim, Seoul (KR); Byung Il Kim, Seoul (KR); Ji Ae Park, Seoul (KR); Sang Keun Woo, Seoul (KR); Kyo Chul Lee, Seoul (KR); Tae Sup Lee, Gangwon-do (KR)

(73) Assignee: KOREA INSTITUTE OF RADIOLOGICAL & MEDICAL SCIENCES, Seoul (KR)

(21) Appl. No.: 14/927,038

(22) Filed: Oct. 29, 2015

(51) Int. Cl. C07K 7/06; A61K 51/08

(52) U.S. Cl. CPC C07K 7/06; A61K 51/08

(57) ABSTRACT

Provided are a novel compound, in which a bombesin derivative known as having selectivity with respect to prostate cancer bonds with a ligand via aminomethyl galacturonic acid, a complex compound that covalently bonds with a radioactive isotope via the ligand of the novel compound, methods of preparing the compounds, and a nuclear-based molecular imaging agent including the complex compound.

### CROSS-REFERENCE TO RELATED APPLICATION

This application relates to Korean Patent Application No. 10-2015-0049464, filed on Apr. 24, 2014, in the Korean Intellectual Property Office, the disclosure of which is incorporated herein in its entirety by reference.

### BACKGROUND

1. Field

One or more exemplary embodiments relate to a novel compound including a bombesin derivative, a method of preparing the novel compound, and a nuclear molecular imaging agent including the novel compound.

2. Description of the Related Art

It has been reported that methods of trying to diagnose and treat cells after labeling a radioactive isotope on a peptide with respect to a receptor existing on a surface of malignant cells, such as tumor cells, are highly selective and efficient in terms of diagnosis and treatment of tumors. Also, the advantages of the peptide for the receptor existing on the malignant cells include high selectivity with respect to normal tissue and fast bonding to the receptor due to a relatively small size of the peptide compared to the size of an antibody.

Studies and developments of the peptides for diagnosis and treatment using a radioactive isotope have been actively conducted worldwide, and representative radioactive isotope labeled peptides that have been developed up to date are shown in Table 1 (Non-patent document 1).

TABLE 1
Progression on clinical and non-clinical studies using a radioactive isotope labeled peptide up to date

| | No. of | | | | | | |
|---|---|---|---|---|---|---|---|
| | amino | | Mechanism of | | Human | Approval | |
| Peptide | acids | Isotopes(s) | binding | Tumor(s) | studies | status | Reference(s) |
| Pentetreotide | 8 | ¹¹¹In | Somatostatin | Carcinoid | Yes | Approved | 2-8 |
| | | | receptor | | | | |
| | | | subtype 2 | | | | |
| DOTATOC | 8 | ⁶⁸Ga | Somatostatin | Carcinoid | Yes | Radioactive | 20, 21 |
| | | | receptor | | | Drug | |
| | | | subtype 2 | | | Research | |
| | | | | | | Committee | |
| | | | | | | approval | |
| DOTATATE | 8 | ⁶⁸Ga | Somatostatin | Carcinoid | Yes | Investigational | |
| | | | receptor | | | New Drug | |
| | | | subtype 2 | | | authorization | |
| Depreotide | 8 | ^{99m}Tc | Somatostatin | Carcinoid | Yes | Approved but | 13-16 |
| | | | receptor | | | no longer | |
| | | | subtype 2 | | | available | |
| α-MSH analog | 13 | ¹¹¹In, ⁶⁸Ga, | MSH receptor | Melanoma | No | Not approved | 24-26 |
| | | ⁸⁶Y, ¹⁸F | | | | | |
| RGD | 3 | ¹⁸F, | Neoangiogenesis | Glioma, | No | Not approved | 27-32 |
| | | ⁶⁴Cu, ¹²⁵I | | melanoma | | | |
| Bombesin | 14 | ¹⁸⁸Re, | Gastrin-releasing | Prostate, | Outside | Not approved | 35-41 |
| | | ^{99m}Tc, | peptide (GRP) | breast | United | | |
| | | ⁶⁴Cu, ¹⁷⁷Lu | receptor | | States | | |
| RGD-bombesin | 3 and | ¹⁸F, ⁶⁴Cu, | Neoangiogenesis | Breast | No | Not approved | 33, 34 |
| dimer | 14 | ⁶⁸Ga | and GRP | | | | |
| | | | receptor | | | | |
| Escherichia coli | 19 | ¹¹¹In | Guanylate | Colorectal | No | Not approved | 42 |
| heat-stable | | | cyclase C | | | | |
| enterotoxin | | | receptor | | | | |
| Vasoactive | 28 | ¹²³I, ⁶⁴Cu | VIP receptor | Prostate | No | Not approved | 43 |
| intestinal | | | | | | | |
| peptide (VIP) | | | | | | | |
| Pituitary | 27 | ^{99m}Tc, ⁶⁴Cu | PACAP receptor | Breast | No | Not approved | 43 |
| adenylate | | | | | | | |
| cyclase- | | | | | | | |
| activating | | | | | | | |
| peptide | | | | | | | |
| (PACAP) | | | | | | | |

Gastrin-releasing peptide (GRP) is one of the peptides having physiological functions such as promoting secretion of gastrin or enzymes from the pancreas and increasing cell proliferation, where a main molecular structure of the peptide is constituted of 27 amino acid residues of which the C-terminal is amidified, and a typical GRP may be a bombesin (BBN)-like family. In mammals, it has been reported that the protein is not released from mucous endocrine cells such as gastrin, but is released from neuron cells. Also, it has been known that GRP stimulates proliferation and infiltration of cancer cells of androgen-independent prostate cancer, an expression of GRP receptor mRNA is measured in 90% of human prostate cancer patients, and GRP receptors are expressed in human prostate cancer cells, such as PC-3, DU-145, and LNCaP cells.

In the non-patent document 2, the current state of studies regarding the diagnosis and treatment of cancer by labeling a radioactive isotope onto a bombesin derivative has been summarized. Producing images of a primary cancer and a bone metastatic cancer by using ^{99m}Tc-RP527 that is prepared by labeling RP527, which is a bombesin-analogues, with ^{99m}Tc in prostate cancer patient, and the diagnosis of metastatic prostate cancer, breast cancer, and benign gastrointestinal stromal tumor by using a [^{99m}Tc]- and [⁶⁸Ga]-labeled BBN peptide has been successful. Also, when a bio-image was obtained by PET after labeling a GRP with ⁶⁸Ga by using a human prostate cancer model in laboratory mice, an uptake rate was as high as 9.5% ID/g. Also, a tumor targeting ability in an animal model for prostate cancer was observed by microSPECT/CT after labeling AMBA (bombesin-like peptide) with a radioactive isotope ¹⁷⁷Lu, and the image of a tumor was obtained by using a molecular imaging technique to measure the distribution of ¹⁷⁷Lu-AMBA. Most clinical trials have been performed by diagnosis using SPECT, and the treatment on the prostate cancer patients was tried by using the radioactive isotope, ¹⁷⁷Lu. The summary of the current state of such studies is as shown in Table 2.

TABLE 2

| | | BBN | Dose | Peptide | | Number of |
|---|---|---|---|---|---|---|
| Researcher | Nuclide | derivative | (MBq) | mass | | patients |
| Van de Wiele | ^{99m}Tc (SPECT) | RP527 | 555 | 3 | ng/kg | 4AI |
| et al. | | | | | | |
| De Vincentis | ^{99m}Tc (SPECT) | [Leu13]BN | 185 | 0.7 | ug | 1AD |
| et al. | | | | | | |
| Scopinaro et al. | ^{99m}Tc (SPECT) | [Leu13]BN | 185 | 0.7 | ug | 8AD |
| De Vincentis | ^{99m}Tc (SPECT) | [Leu13]BN | 185 | 0.7 | ug | 12AD |
| et al. | | | | | | |
| Bodei et al. | ¹⁷⁷Lu (SPECT) | AMBA | 1140-1940 | — | | 7AI |
| Hofmann et al. | ⁶⁸Ga (PET) | DOTABOM | 26-80 | 24 | nmol | 11AD |

AI: androgen independent;
AD: androgen dependent

As stated above, studies on diagnosing and treating cancers by labeling a radioactive isotope on a bombesin derivative have been actively conducted, but most of the peptides for labeling a radioactive isotope has a high liver uptake rate and a low tumor uptake rate, and thus the possibility of misdiagnosis may increase.

### DOCUMENT OF PRIOR ART

### Non-Patent Document

1. Journal of Nuclear Medicine, 52(12), 2011
2. R. P. J. Schroeder et al./Methods 48 (2009) 200-204

### SUMMARY

One or more exemplary embodiments include a novel peptide compound having a low liver uptake ratio and a high selectivity with respect to cancer tissue, the peptide compound is for labeling a radioactive isotope for diagnosing cancer by nuclear molecular imaging.

One or more exemplary embodiments include a method of preparing the novel peptide compound.

One or more exemplary embodiments include a complex compound in which a radioactive isotope for diagnosing cancer is labeled to a novel peptide compound having a low liver uptake ratio and a high selectivity with respect to cancer tissue, wherein the novel compound is for labeling a radioactive isotope for diagnosing cancer by nuclear molecular imaging.

One or more exemplary embodiments include a nuclear molecular imaging agent including the complex compound.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIGS. 1A and 1B show a formulae of NODAGA-BBN and NODAGA-galacto-BBN;

FIGS. 2A and 2B show a formulae of [⁶⁴Cu]NODAGA-BBN and [⁶⁴Cu]NODAGA-galacto-BBN;

FIG. 3 is a high performance liquid chromatography (HPLC) graph of NODAGA-BBN;

FIG. 4 is a mass spectrometry (MS) graph of NODAGA-BBN by using MALDI_TOF;

FIG. 5 is an HPLC graph of NODAGA-galacto-BBN prepared according to an exemplary embodiment;

FIG. 6 is an MS graph of NODAGA-galacto-BBN prepared according to an exemplary embodiment by using MALDI_TOF;

FIG. 7 shows radio thin-layer chromatography (Radio-TLC) images of ⁶⁴Cu, [⁶⁴Cu]NODAGA-BBN, and [⁶⁴Cu]NODAGA-galacto-BBN prepared according to an exemplary embodiment;

FIG. 8 shows a graph of the results of stability measurement performed on [⁶⁴Cu]NODAGA-BBN and [⁶⁴Cu]NODAGA-galacto-BBN in serum of a human and a mouse as prepared according to an exemplary embodiment;

FIG. 9 shows a graph of the results of bonding ability measurements performed on NODAGA-BBN and NODAGA-galacto-BBN prepared according to an exemplary embodiment with respect to a human prostate cancer cell line PC3 cell;

FIG. 10 shows positron emission tomography (PET) scan images taken after injecting [⁶⁴Cu]NODAGA-BBN or [⁶⁴Cu]NODAGA-galacto-BBN prepared according to an exemplary embodiment to a human prostate cancer cell line (PC3 cell) tumor model mouse; and

FIG. 11 shows graphs of liver uptake and tumor/muscle uptake ratios measured from the PET images taken after injecting [⁶⁴Cu]NODAGA-BBN or [⁶⁴Cu]NODAGA-galacto-BBN prepared according to an exemplary embodiment to a human prostate cancer cell line (PC3 cell) tumor model mouse.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an aspect of the present invention, provided is a compound represented by Formula 1 in which a bombesin (BBN) derivative is bonded to a ligand via aminomethyl galacturonic acid.

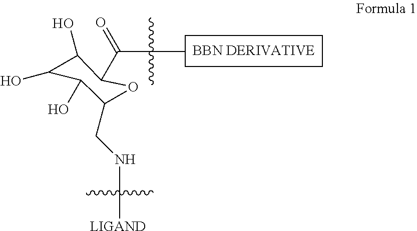

The BBN derivative includes a peptide having a sequence of Gln-Trp-Ala-Val-Gly-His-Leu-Met, where the peptide can bond with a gastrin-releasing peptide (GRP) receptor, and a carboxylic group at the end of the peptide form an amide bond with a carboxylic group of the aminomethyl galacturonic acid.

The ligand is DOTA, DTPA, DO3A, NOTA, NODAGA, TETA, TE3A, TE2A, or PCTA, where a carboxylic group of the ligand forms an amide bond with an amino group of the aminomethyl galacturonic acid.

According to another aspect of an exemplary embodiment, provided is a compound represented by Formula 2 in which a radioactive isotope X is coordinately bonded to the compound of Formula 1:

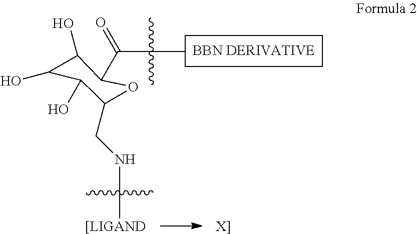

In Formula 2, X is a radioactive isotope that allows SPECT or PET measurements.

According to another aspect of the present invention, provided is a method of preparing the compound of Formula 1 or Formula 2, the method including:

reacting a BBN derivative with aminomethyl galacturonic acid to bond a carboxylic group at the end of the peptide with a carboxylic group of the aminomethyl galacturonic acid; and

reacting the aminomethyl galacturonic acid with a ligand to amide-bond an amino group of the aminomethyl galacturonic acid with a carboxylic group of the ligand.

According to another aspect of the present invention, a SPECT imaging agent for diagnosing cancer includes the compound of Formula 2.

According to another aspect of the present invention, a PET imaging agent for diagnosing cancer includes the compound of Formula 2.

Hereinafter, an exemplary embodiment will be described in detail.

Unless defined otherwise, all technical and scientific terms used herein have the same meanings as commonly understood by one of ordinary skill in the art. Also, protocols and reagents may be used herein to describe particular embodiments, but those similar or equivalent to the protocols and reagents used herein are within the scope the present inventive concept. The contents of all documents used as reference in the present specification are incorporated herein by reference.

The present inventors have studied to resolve the problems of the conventional imaging agent including failure in achieving effective diagnosis and treatment of cancer due to a low selectivity with respect to cancer tissue and a high liver uptake of an imaging agent for diagnosing or treating cancer, wherein the conventional imaging agent is prepared by bonding a ligand to a BBN derivative and then labeling a radioactive isotope to the ligand. As a result, it has been found that when a novel peptide compound was prepared by introducing galacturonic acid as a linker between a BBN derivative and a ligand for labeling a radioactive isotope, the compound labeled with a radioactive isotope appeared to have a high selectivity with respect to cancer cells (Example 3), and that the compound was stable in blood during a sufficient time period needed for nuclear molecular imaging in blood (Example 2). Also, it has been found that when the novel peptide compound was actually administered to a mouse of prostate cancer model and the PET image of the model was taken, the compound had a relatively high tumor uptake rate and a relatively low liver uptake compared to those of a case that does not include galacturonic acid as a linker (Example 4).

Therefore, according to an aspect of the present invention, provided is a compound represented by Formula 1, in which a BBN derivative is bonded to a ligand via aminomethyl galacturonic acid:

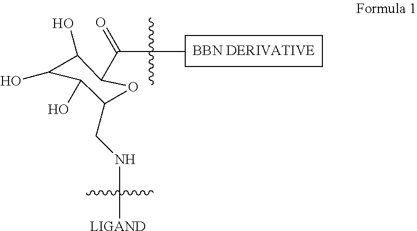

The BBN derivative includes a peptide having a sequence of Gln-Trp-Ala-Val-Gly-His-Leu-Met, where the peptide can be bonded to a GRP receptor, and a carboxylic group at the end of the peptide form an amide bond with a carboxylic group of the aminomethyl galacturonic acid.

The ligand is DOTA, DTPA, DO3A, NOTA, NODAGA, TETA, TE3A, TE2A, or PCTA, where a carboxylic group of the ligand forms an amide bond with an amino group of the aminomethyl galacturonic acid.

The compound of Formula 2 may be prepared by forming a complex by bonding a radioactive isotope for nuclear molecular imaging to the ligand of the compound of Formula 1. Thus, according to another aspect of the present invention, provided is the compound represented by Formula 2:

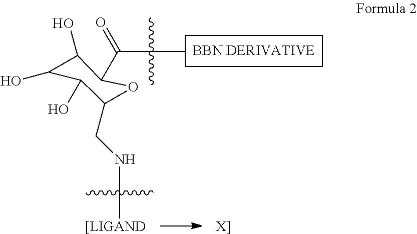

In Formula 2,

the BBN derivative and the ligand are the same as defined in connection with Formula 1, and

X is a radioactive isotope that allows SPECT or PET measurements and is coordinately bonded to the ligand.

As used herein, the expression “ligand→X” refers to a complex that is formed by coordinately bonding a radioactive isotope X to the ligand.

As used herein, the expression “complex” refers to an atom group consisting of a central atom or ion, which is usually metallic and is called the coordination centre, and a surrounding array of bound molecules or ions, that are in turn known as ligands. Here, a chelator coordinated with the central atom or ion is referred to as a ligand.

As used herein, the term “DOTA” refers to 1,4,7,10-tetraazacyclododecane-1,4,7,10-tetraacetic acid, the term “DTPA” refers to diethylene triamine pentaacetic acid, the term “DO3A” refers to 1,4,7,10-tetraazacyclododecane-1,4,7-triacetic acid, the term “NOTA” refers to 1,4,7-triazacyclononane-1,4,7-triacetic acid, the term “NODAGA” refers to 1,4,7-triazacyclononane,1-glutaric acid-4,7-acetic acid, the term “TETA” refers to 1,4,8,11-tetraazacyclotetradecane-N,N′,N″,N′″-tetraacetic acid, the term “TE3A” refers to 1,4,8,11-tetraazacyclotetradecane-1,4,8-triacetic acid, the term “TE2A” refers to 1,4,8,11-tetraazabicyclohexadecane-4,11-diacetic acid, the term “PCTA” refers to 3,6,9,15-tetraazabicyclo[9.3.1]pentadeca-1,11,13-triene-3,6,9-triacetic acid, and the acids are ligand compounds having metal affinity. The ligand acts as a metal affinity ligand so that a radioactive isotope in the body may not be dissociated. Also, the ligand discharges a radioactive material to outside the body so that cell toxicity caused by the radioactive material may be reduced, and thus the ligand may act as a chemical protective agent with respect to radiation hazards (Marouan Rami et al., Carbonic anhydrase inhibitors: Gd(III) complexes of DOTA- and TETA-sulfonamide conjugates targeting the tumor associated carbonic anhydrase isozymes IX and XII, New J. Chem., 2010, 34, 2139-2144; Silvio Aime et al., NMR relaxometric studies of Gd(III) complexes with heptadentate macrocyclic ligands, Magnetic Resonance in Chemistry (1998) Volume: 36, Issue: S1, Pages: S200-S208).

As used herein, the radioactive isotope “X” is a radioactive isotope for nuclear molecular imaging, or, particularly, refers to an arbitrary isotope that allows SPECT or PET measurements. For example, X may be a radioactive iodine selected from I-123, I-124, I-125, and I-131, Tc-99m, Re-188, Re-186, or Lu-177 as an isotope for SPECT measurement; or Cu-64, Cu-67, Ga-68, or Zr-89 as an isotope for PET measurements. In some embodiments, the radioactive isotope is ⁶⁴Cu.

As used herein, the term “a BBN derivative” includes any peptides that include a peptide of a Gln-Trp-Ala-Val-Gly-His-Leu-Met sequence and bond to a GRP receptor. The Gln-Trp-Ala-Val-Gly-His-Leu-Met sequence is an active fragment of BBN, and the BBN derivative is known to be able to specifically bond to prostate cancer, breast cancer, small cell lung cancer, stomach cancer, or neuroblastoma cells by including the active fragment of BBN (Non-patent document 2). The BBN derivative may target cancer tissue by bonding with a GRP receptor that is expressed in cells of specific cancer but not expressed in normal tissue, such as prostate cancer or breast cancer, and thus the compound of Formula 2 may act as an effective imaging agent for diagnosing cancer.

When the conditions described above are satisfied, the BBN derivative may include additional amino acid sequences in addition to the Gln-Trp-Ala-Val-Gly-His-Leu-Met sequence. One of ordinary skill in the art may prepare a BBN derivative that satisfies the conditions described above in view of common knowledge in the art. In some embodiments, the BBN derivative consists of a peptide of the Gln-Trp-Ala-Val-Gly-His-Leu-Met sequence.

In some embodiments, the compound of Formula 2 may be a compound represented by Formula 2a:

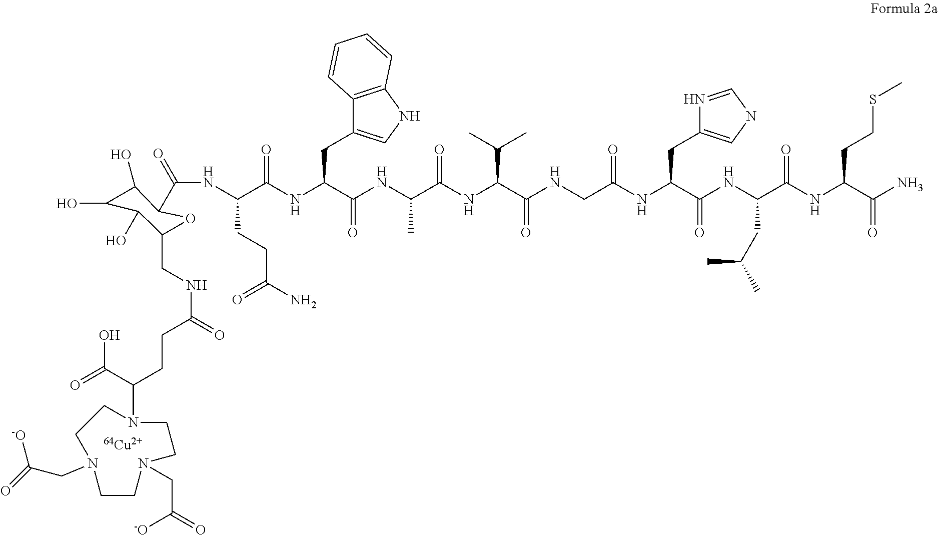

According to another aspect of an exemplary embodiment, provided is a method of preparing the compound of Formula 1 or Formula 2, the method including:

reacting a BBN derivative with aminomethyl galacturonic acid to amide-bond a carboxylic group at the end of a peptide and a carboxyl group of the aminomethyl galacturonic acid; and

reacting the aminomethyl galacturonic acid with a ligand to amide-bond an amino group of the aminomethyl galacturonic acid and a carboxyl group of the ligand.

The BBN derivative and the ligand are the same as defined in connection with Formula 1 or Formula 2.

The amide-bonding of the BBN derivative and the aminomethyl galacturonic acid may be performed by one of ordinary skill in the art of organic chemistry. Also, the amide-bonding of the aminomethyl galacturonic acid and the ligand may be performed by one of ordinary skill in the art of organic chemistry.

The BBN derivative that constitutes the compound of Formula 1 or Formula 2 may be prepared by peptide-bonding an amino acid by Fmoc solid phase peptide synthesis known in the art. The preparation of a peptide by the Fmoc solid phase peptide synthesis is known in the art, and thus the BBN derivative may be prepared by selecting appropriate reaction conditions.

The compound of Formula 2 may be prepared by reacting the compound of Formula 1 and a radioactive isotope together to form a complex by bonding the radioactive isotope with a ligand of the compound of Formula 1. Also, the compound of Formula 1 may be prepared by the Fmoc solid phase peptide synthesis. In particular, the compound of Formula 1 may be prepared by using a Fmoc solid phase peptide synthesis method using aminomethyl galacturonic acid, a ligand, and amino acid units as reactants.

As a result of the experiment, the compound of Formula 2 labeled with a radioactive isotope appeared to have a high selectivity with respect to a cancer cell (Experiment 3), and that the compound was stable in blood during a sufficient time period needed for nuclear molecular imaging in blood (Example 2). Also, it has been found that when the novel peptide compound was actually administered to a mouse of prostate cancer model and the PET image of the model was taken, the compound had a relatively high tumor uptake rate and a relatively low liver uptake compared to those of a case that does not include galacturonic acid as a linker (Example 4). Therefore, it was found that the compound of Formula 2 may be effectively used as a nuclear molecular imaging agent for SPECT or PET.

Thus, according to another aspect of an exemplary embodiment, provided is a SPECT imaging agent including the compound of Formula 2 or Formula 2a.

Thus, according to another aspect of an exemplary embodiment, provided is a PET imaging agent including the compound of Formula 2 or Formula 2a.

The SPECT imaging agent or the PET imaging agent are selective in a cancer cell expressing a GRP receptor, and thus the imaging agents may be used in diagnosis of breast cancer or prostate cancer known as expressing a GRP receptor. Also, the imaging agents has a high tumor uptake rate with respect to a liver and a low liver uptake rate and thus may be used as effective imaging agents in diagnosing a tumor.

A dose of the SPECT imaging agent administered to an adult may be in a range of about 0.5 mCi/kg to about 1 mCi/kg based on the compound of Formula 2, which is an active ingredient.

A dose of the PET imaging agent administered to an adult may be in a range of about 0.5 mCi/kg to about 1 mCi/kg based on the compound of Formula 2, which is an active ingredient

The SPECT imaging agent or the PET imaging agent may be formulated into an injection, and, in this case, a non-toxic buffer solution that is isotonic with blood may be used as a diluting agent. An example of the non-toxic buffer solution may be a phosphoric acid buffer solution of pH 7.4. The SPECT or PET imaging agent may include other diluting agents or additives in addition to the buffer solution. The diluting agents or additives that may be added to the injection are known in the art, and, for example, may be known in light of the following document (Dr. H. P. Fiedler “Lexikon der Hilfsstoffe fur Pharmazie, Kosmetik und angrenzende Gebiete” [Encyclopedia of auxiliaries for pharmacy, cosmetics and related fields]).

Thereinafter, one or more embodiments of the present invention will be described in detail with reference to the following examples. However, these examples are not intended to limit the scope of the one or more embodiments of the present invention.

### DEFINITION OF ABBREVIATIONS IN EXAMPLES

NODAGA-BBN: A compound formed by bonding NODAGA to a Gln-Trp-Ala-Val-Gly-His-Leu-Met peptide, which is an active fragment of a bombesin peptide

NODAGA-galacto-BBN: A compound formed by bonding NODAGA to a Gln-Trp-Ala-Val-Gly-His-Leu-Met peptide via an aminomethyl galacturonic acid a linker, which is an active fragment of a bombesin peptide

[⁶⁴Cu]NODAGA-galacto-BBN: A complex including ⁶⁴Cu coordinate-bonded to NODAGA of NODAGA-galacto-BBN

SPPS: Solid-phase peptide synthesis

HPLC: High performance liquid chromatography

TFA: Trifluoroacetic acid

ACN: Acetonitrile

RT: Retention time

Radio TLC: Radio thin-layer chromatography

BSA: Bovine serum albumin

### Preparation Example 1: Preparation of NODAGA-BBN and NODAGA-Galacto-BBN

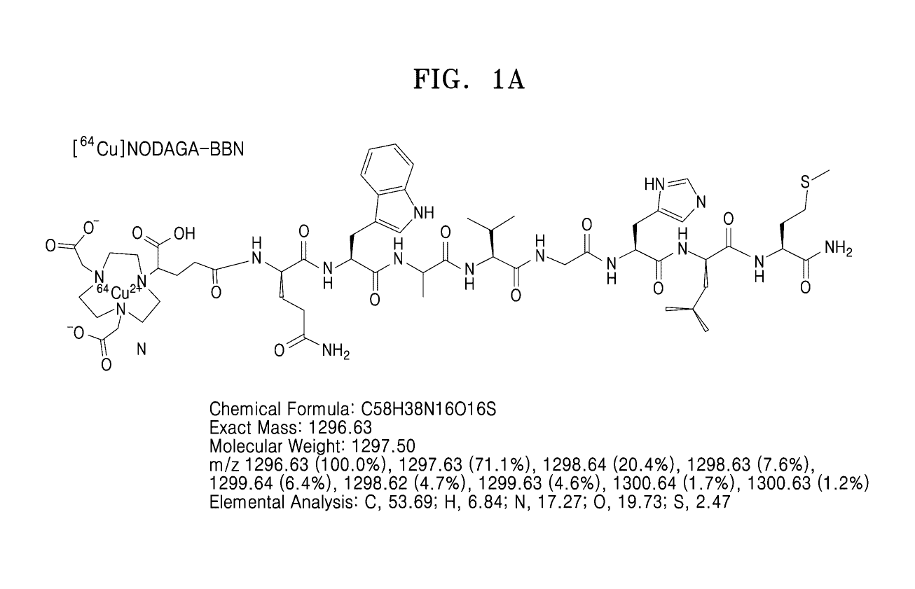

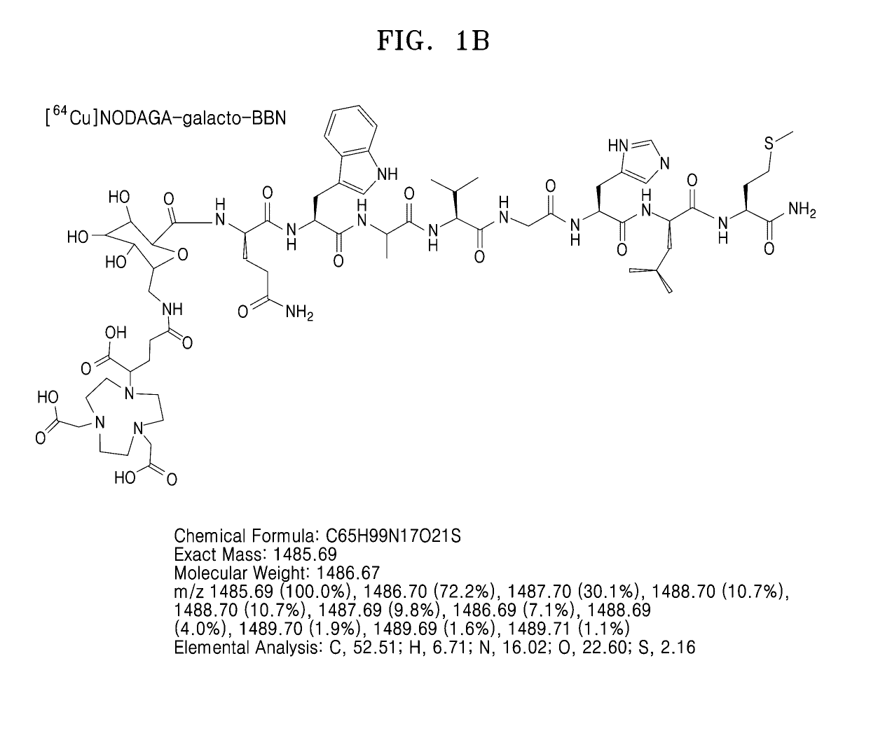

NODAGA-BBN and NODAGA-galacto-BBN having structures as shown in FIG. 1 were synthesized by using a Fmoc-based SPPS using a PIT-symphony peptide synthesis synthesizer.

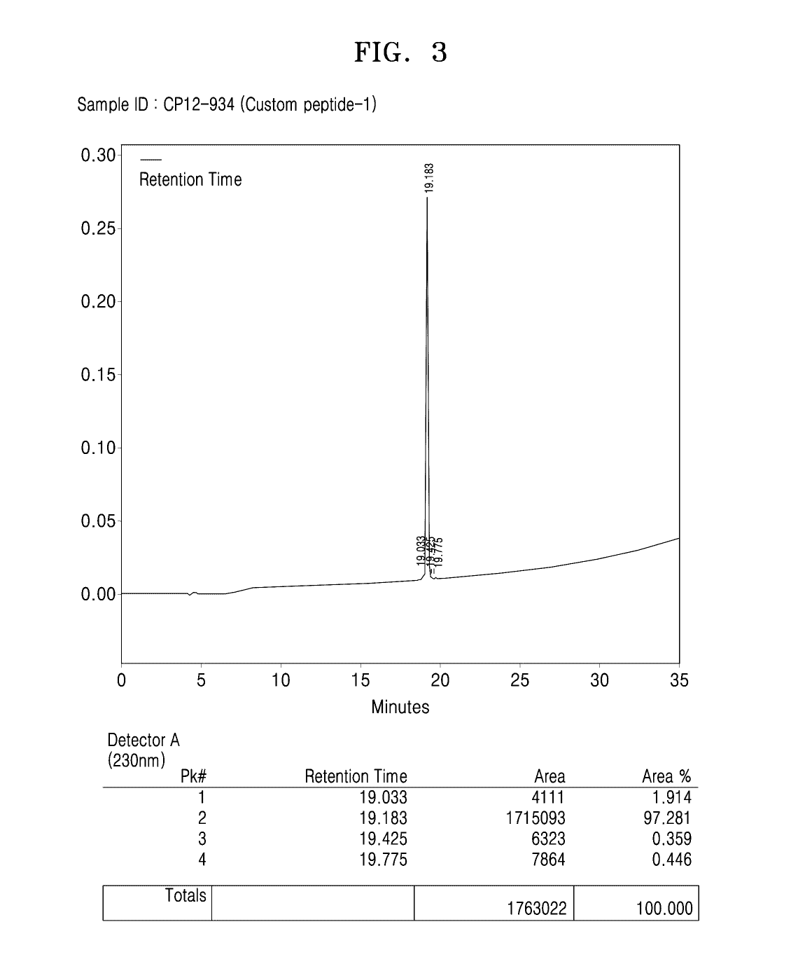

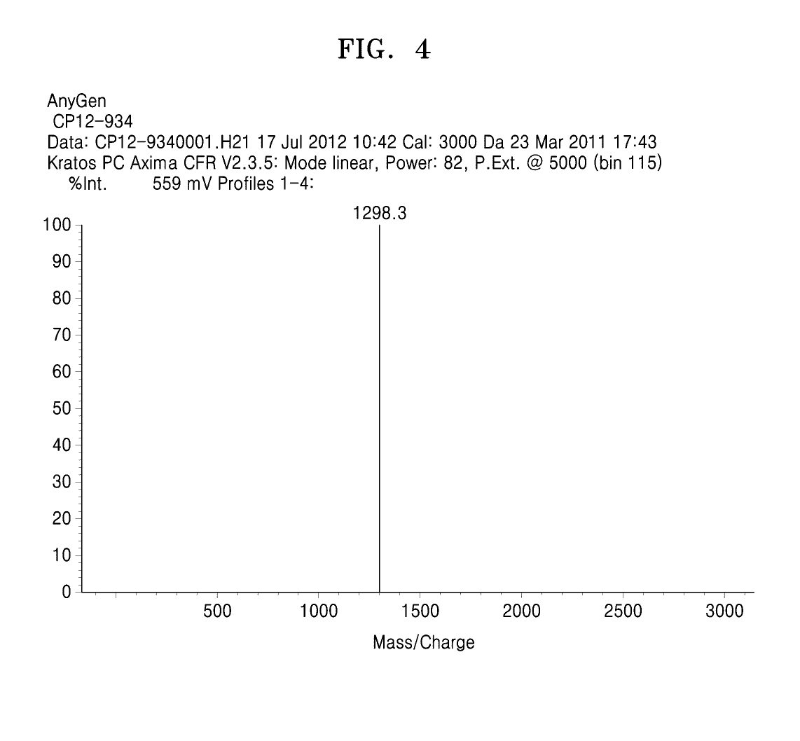

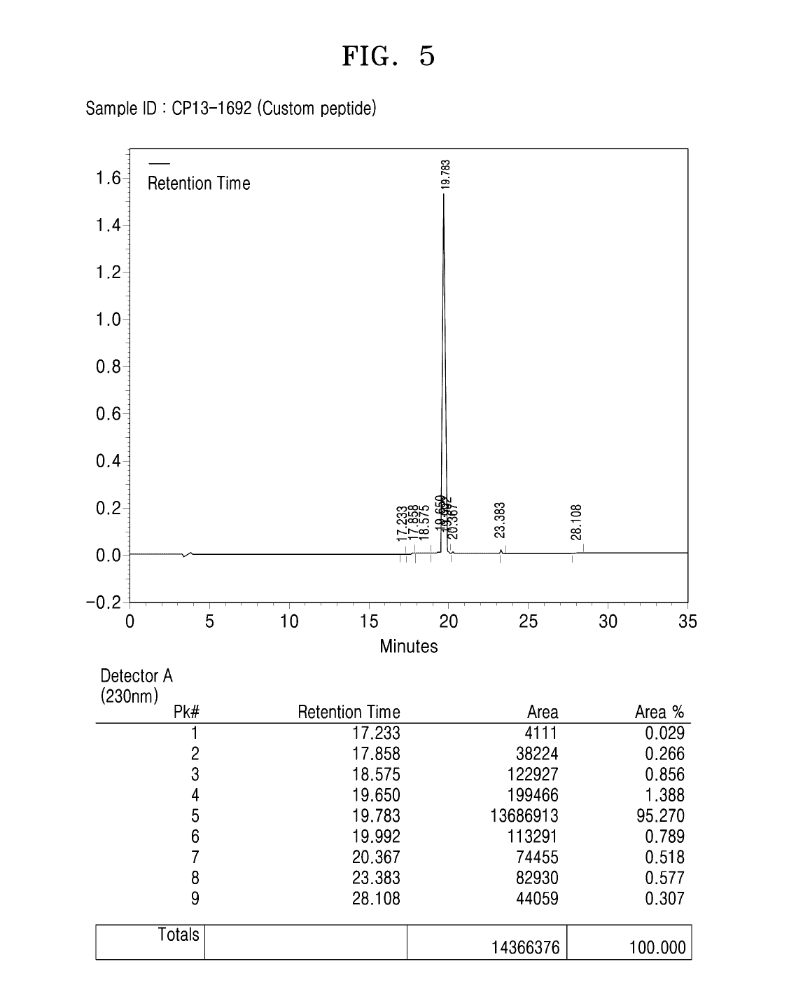

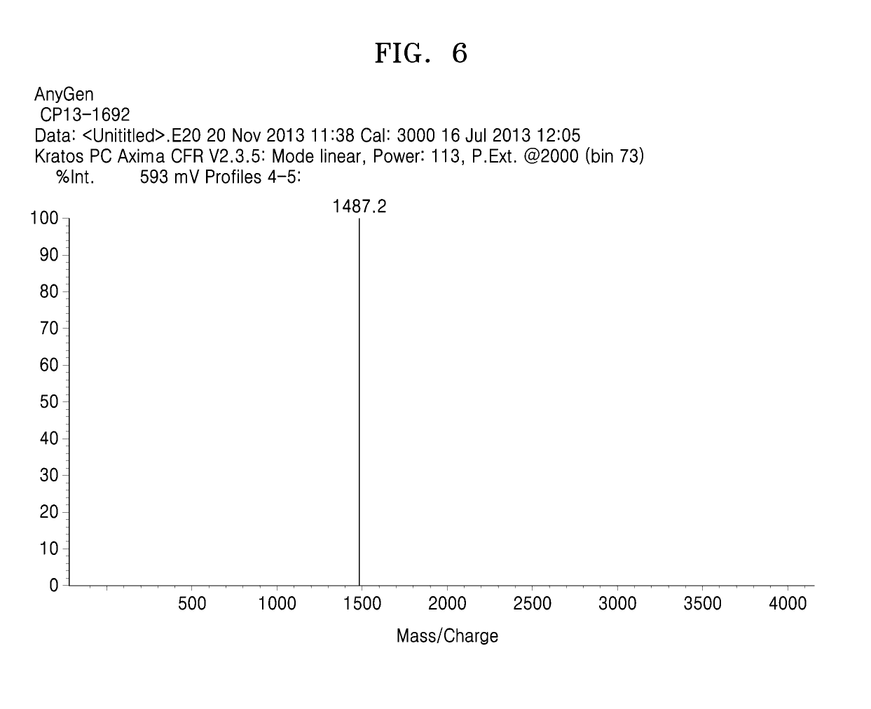

NODAGA-BBN and NODAGA-galacto-BBN thus prepared were analyzed by HPLC. When a 0.1% TFA aqueous solution (solution A) and a 0.1% TFA solution in ACN (solution B) were flowed through a SHIMADZU C-18 analytical column (10.0 mm×250 mm) at a B composition of about 5% to about 65% for 30 minutes at a rate of 1 mL/min, and thus peaks were observed at RT 19.183 minute (NODAGA-BBN) and RT 19.783 minute (NODAGA-galacto-BBN). The results are shown in FIGS. 3 and 5. Also, the prepared NODAGA-BBN and NODAGA-galacto-BBN were analyzed by MALDI-TOE-MS, and the results are shown in FIGS. 4 and 6.

FIG. 3 shows an HPLC image of the prepared NODAGA-BBN, and FIG. 4 shows an MALDI-TOF-MS image of the prepared NODAGA-BBN.

FIG. 5 shows an HPLC image of the NODAGA-galacto-BBN prepared according to an exemplary embodiment, and FIG. 6 shows an MALDI-TOF-MS image of the NODAGA-galacto-BBN prepared according to an exemplary embodiment.

### Preparation Example 2: Preparation of [⁶⁴Cu]NODAGA-BBN and [⁶⁴Cu]NODAGA-Galacto-BBN

24 μL of a NODAGA-BBN or NODAGA-galacto-BBN solution prepared by dissolving the NODAGA-BBN or NODAGA-galacto-BBN prepared in Preparation Example 1 in 1 M sodium acetic acid at a concentration of 1 mg/mL was mixed with 1 mL of a sodium acetate buffer solution of pH 4. Preparation of the sodium acetate buffer solution of pH 4 was made by, first, preparing 1 L of stock solution A [prepared by diluting 11.55 ml of glacial acetic acid (CH₃COOH) in distilled water to a concentration of 0.2 M] and 1 L of stock solution B [prepared by diluting 16.41 g of anhydrous sodium acetate (CH₃COONa) or 7.22 g of CH₃COONa-3H₂O in distilled water to a concentration of 0.2 M], and mixing 296 μL of the stock solution A and 704 μL of the stock solution B. Then, the mixture was reacted with ⁶⁴Cu (pH 4 sodium acetate buffer/200 μL) of 9.2-74 MBq (0.5-2 mCi) at a temperature of 70° C. for 10 minutes. After the reaction, a radiochemical yield of the final product was measured by radio-TLC.

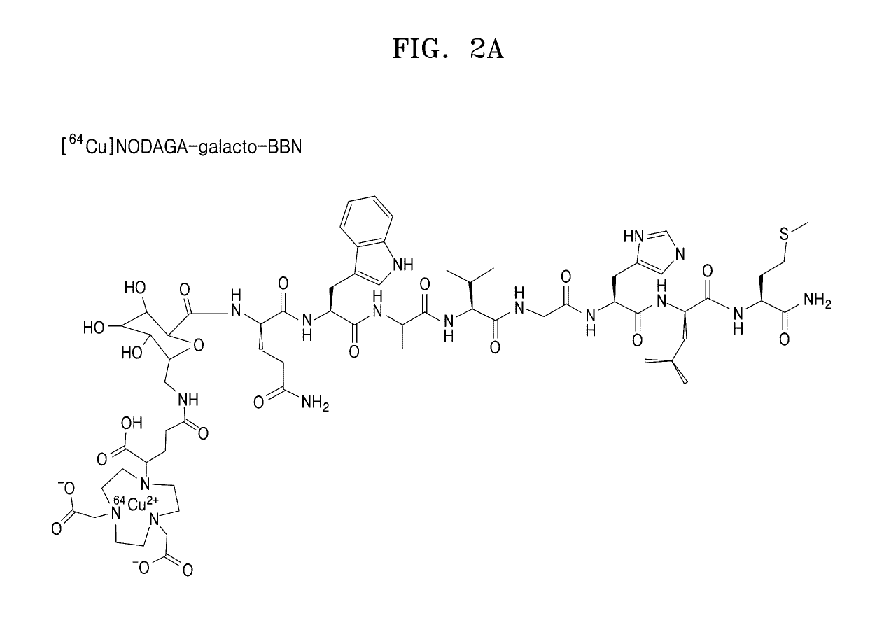

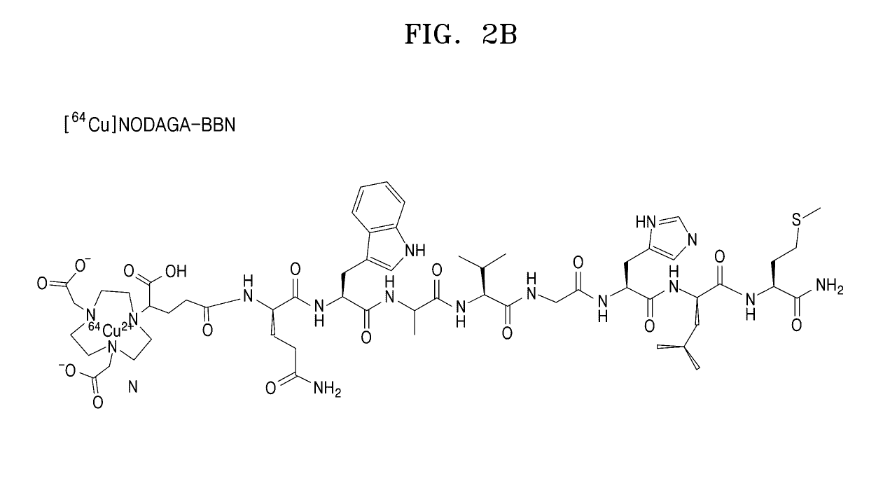

Structures of the [⁶⁴Cu]NODAGA-BBN and [⁶⁴Cu]NODAGA-galacto-BBN thus prepared are shown in FIG. 2.

### Experimental Example 1: Measurement of Radiochemical Yield of NODAGA-BBN and NODAGA-Galacto-BBN by Using ⁶⁴Cu

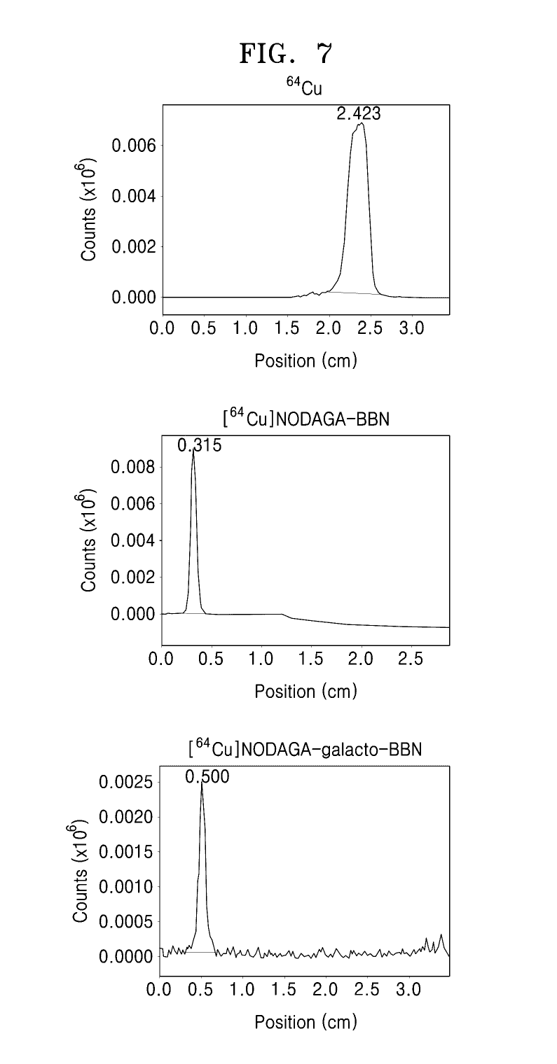

Radiochemical yields of the [⁶⁴Cu]NODAGA-BBN and [⁶⁴Cu]NODAGA-galacto-BBN prepared in Preparation Example 2 were analyzed by radio TLC, and the results are shown in FIG. 7.

As shown in FIG. 7, it was found that a radiochemical yield of the [⁶⁴Cu]NODAGA-BBN and [⁶⁴Cu]NODAGA-galacto-BBN after the reaction with ⁶⁴Cu at a temperature of 70° C. for 10 minutes was 100%.

### Example 2: Test for Stability of [⁶⁴Cu]NODAGA-BBN and [⁶⁴Cu]NODAGA-Galacto-BBN in Serum

In order to test stability in serum, human serum, mouse serum, saline solution, and [⁶⁴Cu]NODAGA-BBN or [⁶⁴Cu]NODAGA-galacto-BBN were mixed, and while allowing a reaction to occur therein at a temperature of 37° C. for 24 hours, stability of the final product was measured at specific time points by radio-TLC.

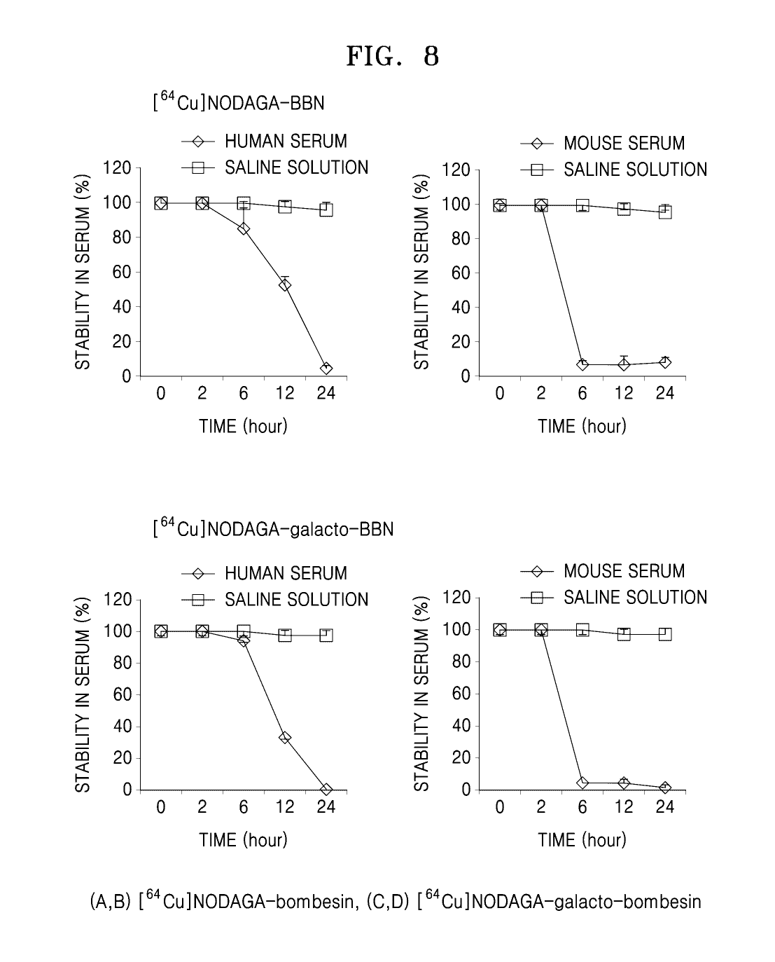

The results are shown in FIG. 8.

According to data shown in FIG. 8, it was found that [⁶⁴Cu]NODAGA-BBN and [⁶⁴Cu]NODAGA-galacto-BBN were stable for 2 hours in human serum and mouse serum, and for 24 hours in saline solution.

### Example 3: Comparison of Cell Bonding Ability with Respect to Human Prostate Cancer Cell Line (PC3)

Bonding abilities of the NODAGA-BBN and NODAGA-galacto-BBN synthesized in Preparation Example 1 with respect to PC3 cells were compared. The control group was [¹²⁵I]Try⁴-BBN (Perkinelmer Co. MA).

0.06 nM [¹²⁵I]Try⁴-BBN (NEX258050UC, PerkinElmer Co. MA) and a material (NODAGA-BBN or NODAGA-galacto-BBN) at a various concentration of about 1.00-E4 to about 1.00-E13 M were added to a binding buffer with PC3 cells (2×10⁶), and contents in the mixture were allowed to react for 1 hour while stirring the mixture at room temperature. The binding buffer was prepared by mixing 25 mM Tris at pH 7.4, 150 mM NaCl, 1 mM MnCl₂, and 0.1% BSA (bovine serum albumin). When the reaction was completed, the resultant was twice washed with 3 mL of phosphate buffer saline (PBS), and the radioactivity remained in each tube was measured by using the Gamma counter (PerkinElmer Co. MA). An IC₅₀ value was obtained by nonlinear regression using GrasphPad Prism (GraphPad Software, Inc., CA).

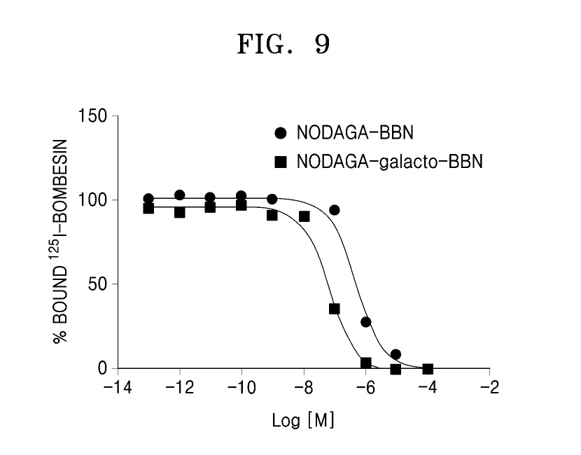

The results are shown in FIG. 9.

Referring to FIG. 9, the IC₅₀ values of NODAGA-BBN and NODAGA-galacto-BBN were (5.47±0.38)×10⁷ mol/L and (6.67±1.07)×10⁸ mol/L, respectively. Thus, it was confirmed that a binding ability to PC3 cells of NODAGA-galacto-BBN was significantly higher than that of NODAGA-BBN.

### Example 4: Obtain PET Image In Vivo of Human Prostate Cancer Cell Line (PC3) Tumor Model Mouse

A target effect of the [⁶⁴Cu]NODAGA-BBN or [⁶⁴Cu]NODAGA-galacto-BBN prepared in Preparation Example 2 with respect to a human prostate cancer cell line (PC3) tumor model was tested. A human prostate cancer cell line (PC3) tumor model was prepared by using BALB/c-nu/nu mice (male, about 6-week old, weight: about 20 g to about 25 g) (NarabioTec, Seoul, Korea) or NOD.CB17-Prkdc^{scid} mice (male, about 6-week old, weight: about 20 g to about 25 g). The human prostate cancer cell line (PC3) at a cell concentration of 5×10⁶ was subcutaneously injected into the left or right hind leg of the mice to prepare a human prostate cancer cell line (PC3) tumor model.

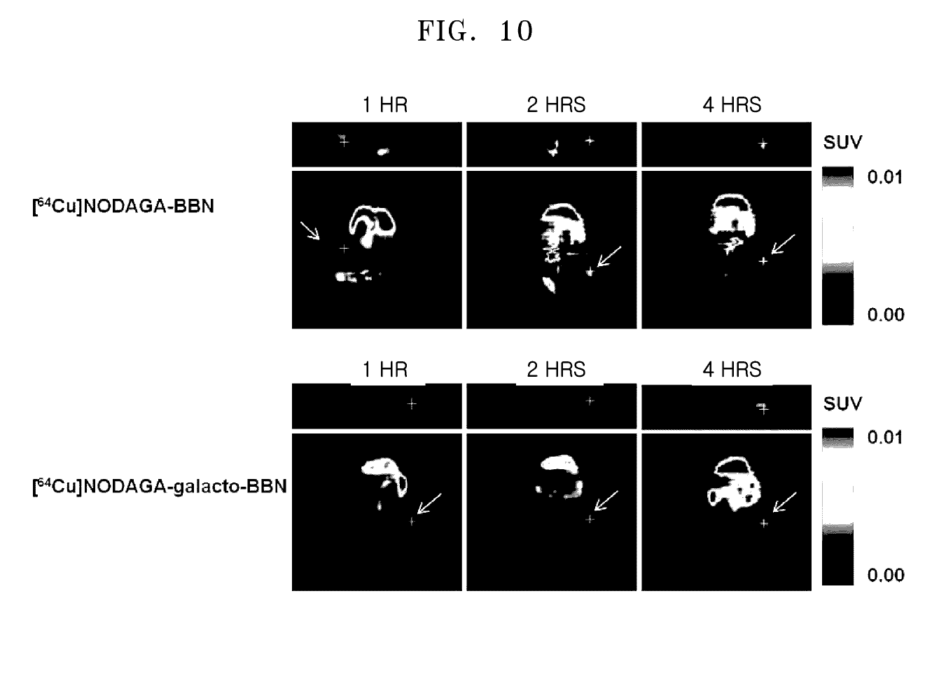

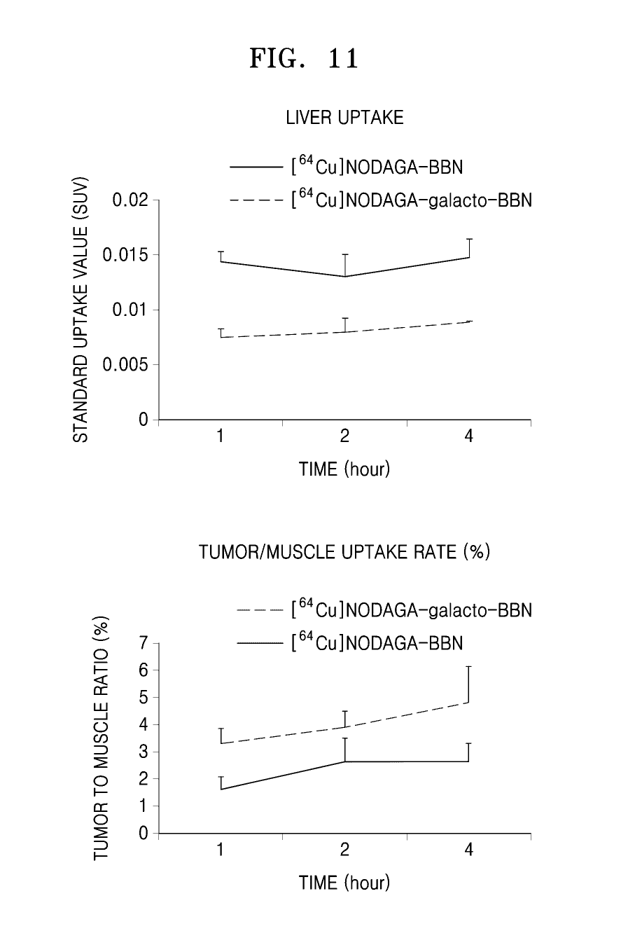

After performing inhalation anesthesia with 2% isofluorane, the mice was intravenously injected with 16.7 to 18.5 MBq (450 to 500 μCi) of [⁶⁴Cu]NODAGA-BBN or [⁶⁴Cu]NODAGA-galacto-BBN via tail vein of the mice. Then, PET images were taken for 60 minutes after 1, 2, and 4 hours from the injection. The results are shown in FIG. 10.

As shown in FIG. 10, the images taken after 1, 2, and 4 hours from the injection confirmed that the group of mice injected with [⁶⁴Cu]NODAGA-galacto-BBN had a significantly low liver uptake than that of the group of mice injected with [⁶⁴Cu]NODAGA-BBN. Also, the images taken after 1, 2, and 4 hours from the injection confirmed that the group of mice injected with [⁶⁴Cu]NODAGA-galacto-BBN had a significantly high tumor to muscle ratio than that of the group of mice injected with [⁶⁴Cu]NODAGA-BBN.

As described above, according to the one or more of the above embodiments of the present invention, a compound represented by Formula 2 or Formula 2a contains a bombesin derivative, which has a high affinity to a GRP receptor, and thus the compound may be used as an imaging agent for nuclear molecular imaging that is specific to a particular cancer such as breast cancer or prostate cancer and safe. Also, since the compound of Formula 2 or 2a includes a structure of galacturonic acid, a liver uptake ratio decreases, and thus a cancer may be accurately diagnosed.

1. A compound of Formula 1, in which a bombesin (BBN) derivative bonds with a ligand via aminomethyl galacturonic acid: 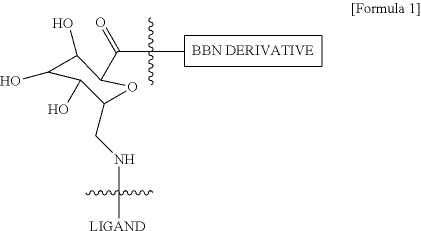
wherein, in Formula 1, the bombesin (BBN) derivative comprises a peptide having a sequence of Gln-Trp-Ala-Val-Gly-His-Leu-Met (SEQ ID NO: 1), wherein the peptide can bond with a gastrin-releasing peptide (GRP) receptor, wherein an amino group at an end of the peptide forms an amide bond with a carboxyl group of the aminomethyl galacturonic acid,
wherein the ligand is DOTA, DTPA, DO3A, NOTA, NODAGA, TETA, TE3A, TE2A, or PCTA, wherein a carboxyl group of the ligand forms an amide bond with an amino group of the aminomethyl galacturonic acid.

2. The compound of claim 1, wherein the BBN derivative is Gln-Trp-Ala-Val-Gly-His-Leu-Met (SEQ ID NO: 1), and the ligand is NODAGA.

3. A compound of Formula 2, in which a radioactive isotope X is coordinately bonded to the ligand of the compound of Formula 1 according to claim 1 or 2: 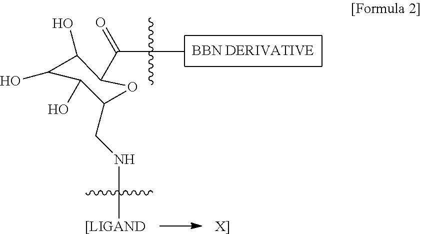
wherein, in Formula 2, X is a radioactive isotope that enables measurement of SPECT or PET.

4. The compound of claim 3, wherein the radioactive isotope enabling measurement of SPECT is an iodide selected from I-123, I-124, I-125, and I-131, Tc-99m, Re-188, Re-186, or Lu-177.

5. A SPECT imaging agent for diagnosing cancer, the SPECT imaging agent comprising the compound of claim 4.

6. The SPECT imaging agent of claim 5, wherein the cancer is prostate cancer, breast cancer, small cell lung cancer, gastric cancer, or neuroblastoma.

7. The compound of claim 3, wherein the radioactive isotope that enables measurement of PET is Cu-64, Cu-67, Ga-68, or Zr-89.

8. The compound of claim 7, having a structure of Formula 2a:

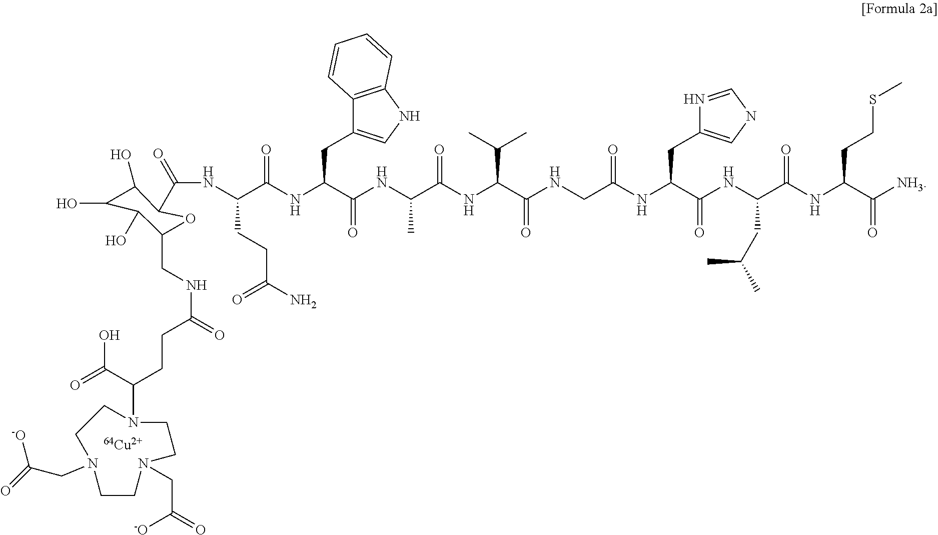

9. A PET imaging agent for diagnosing cancer, the PET imaging agent comprising the compound of claim 8.

10. The PET imaging agent of claim 9, wherein the cancer is prostate cancer, breast cancer, small cell lung cancer, gastric cancer, or neuroblastoma.

11. A method of preparing the compound of claim 1, the method comprising:
reacting a BBN derivative with aminomethyl galacturonic acid so that an amino group at the end of the peptide amide-bonds with a carboxyl group of the aminomethyl galacturonic acid; and
reacting the aminomethyl galacturonic acid with a ligand to amide-bond an amino group of the aminomethyl galacturonic acid and a carboxyl group of the ligand,
wherein the BBN derivative and the ligand are the same as defined in connection with claim 1.
